(19) United States
(12) Patent Application Publication
(10) Pub. No.: US 2021/0037296 A1
(43) Pub. Date: Feb. 4, 2021

(54) CONCRETE MONITORING AND ANALYSIS SYSTEMS AND METHODS

(71) Applicant: FLIR Systems, Inc., Wilsonville, OR (US)

(72) Inventors: James O'Daniel, Stillwater, OK (US); Dale Gillham, Orlando, FL (US)

(21) Appl. No.: 16/528,561

(22) Filed: Jul. 31, 2019

(51) Int. Cl. H04Q 9/00; H04L 29/08; G01N 33/38

(52) U.S. Cl. CPC H04Q 9/00; H04L 67/12; H04Q 2209/82; H04Q 2209/40; G01N 33/383

(57) ABSTRACT

Systems and methods for monitoring and reporting data associated with a concrete mass include communications components configured to receive concrete data from at least one logger associated with a concrete mass, a storage device configured to store the received concrete data, and a logic device configured to detect an event in the concrete data, transmit a notification of the detected event to at least one remote device, render an interface for the remote device, the interface facilitating visualization of the stored concrete data. The communications components may be configured to receive the concrete data from a reader device and distributed the logger data to the at least one remote device. The interface may comprise an interactive control facilitating user adjustment of comparison points for multiple loggers.

### TECHNICAL FIELD

The present application relates generally to concrete monitoring and analysis and, more particularly for example, to systems and methods for sensing, storing, analyzing and/or reporting data associated with concrete and other construction materials.

### BACKGROUND

Concrete is one of the most widely used construction materials. The term “concrete” is generally used in the construction industry to refer to a mixture of Portland cement or other cementitious or pozzolanic materials, coarse aggregate such as gravel, fine aggregate such as sand, water and various chemical admixtures which, upon hydration of the cementitious and pozzolanic materials, becomes a hardened mass.

When used in construction projects, it is desirable to know the strength of the concrete during the curing process. Curing may be understood as the strengthening of the concrete through the process of hydration that occurs over a number of days. When concrete stays moist, the moisture allows the chemical reaction between the cementitious materials and water to continue. From the curing temperature history, one can determine the mechanical strength from previously-determined empirical equations. One method for determining the strength of a mass of concrete is the “maturity method.” In the maturity method, a record of the internal temperature history of the concrete mass is collected as the concrete cures. U.S. Pat. No. 7,970,554 discloses methods and devices for securely storing data relating to concrete maturity, concrete strength and other concrete data.

There is a continued need for improved systems and methods for non-destructive testing of concrete masses (e.g., using the maturity method) and analyzing data to shorten construction schedules, promote safety and save money.

### SUMMARY

Embodiments of the present disclosure relate generally to recording time-stamped activity data associated with a concrete mass, storing temperature data within a protected environment of a concrete mass, and securely calculating and storing data relating to the properties of a concrete mass and, more specifically, mechanical strength data of the concrete throughout its curing process.

In one or more embodiments, a system comprises communications components configured to receive concrete data from at least one logger associated with a concrete mass, a storage device configured to store the received concrete data, and a logic device configured to detect an event in the concrete data, transmit a notification of the detected event to at least one remote device, render an interface for the remote device, the interface facilitating visualization of the stored concrete data. The communications components may be configured to receive the concrete data from a reader device and distributed the logger data to the at least one remote device. The interface may comprise an interactive control facilitating user adjustment of comparison points for multiple loggers.

The logger may comprise at least one sensor adapted to measure a property of the concrete mass and generate sensor data indicative of the measured property, a logic device configured to receive the sensor data and calculate data associated with the concrete mass, and a data transfer device configured to transmit the sensor data to an external device. A reader is configured to receive the logger data associated with the concrete mass from the data transfer device of the logger and may comprise an infrared imaging component configured to capture an infrared image of the concrete mass; and wherein the concrete data includes the captured infrared image. The logger may be further configured to store non-sensor data comprising location information, a project identifier, time-related data, mix design information, temperature, activation energy and/or a threshold value.

In some embodiments, the logger data comprises temperature, moisture, relative humidity, electrical conductivity, thermal conductivity, alkalinity, oxidation-reduction potential and/or sonic velocity. The storage device may be implemented through a cloud storage device and the logic device may comprise a cloud application server. The logger may be further configured to capture and store sensed data approximately every minute and the communications components are configured to receive concrete data at the cloud storage device after the concrete data is available from the logger, thereby providing a remote device with up-to-the minute access to the concrete data.

In one or more embodiments, a method comprises receiving, through communications components, concrete data from at least one logger associated with a concrete mass, storing the received concrete data in a storage device, detecting an event in the concrete data, transmitting a notification of the detected event to at least one remote device, and rendering an interface for the remote device, the interface facilitating visualization of the stored concrete data. The method may further comprise measuring a property of the concrete mass using a sensor associated with the logger and generating sensor data indicative of the measured property, receiving the sensor data and calculating data associated with the concrete mass, and transmitting the sensor data and the calculated data from the logger to an external device. The concrete data may be received at a reader device and then distributed to at least one remote device. The interface may comprise an interactive control facilitating user adjustment of comparison points for multiple loggers.

The method may further comprise capturing an infrared image of the concrete mass, wherein the concrete data includes the captured infrared image, and/or storing non-sensor data on the logger including location information; a project identifier, time-related data, mix design information, temperature, activation energy and/or a threshold value. The concrete data may comprise temperature, moisture, relative humidity, electrical conductivity, thermal conductivity, alkalinity, oxidation-reduction potential and/or sonic velocity.

In some embodiments, the storage device is a cloud storage device and the method further comprises generating a cloud application interface. The method may further comprise capturing and storing sensed data approximately every minute and receiving concrete data at a cloud service after the concrete data is available from the logger, thereby providing a remote device with up-to-the minute access to the concrete data.

The scope of the present disclosure is defined by the claims, which are incorporated into this section by reference. A more complete understanding of embodiments of the invention will be afforded to those skilled in the art, as well as a realization of additional advantages thereof, by a consideration of the following detailed description of one or more embodiments. Reference will be made to the appended sheets of drawings that will first be described briefly.

### BRIEF DESCRIPTION OF THE DRAWINGS

Aspects of the disclosure and their advantages can be better understood with reference to the following drawings and the detailed description that follows. It should be appreciated that like reference numerals are used to identify like elements illustrated in one or more of the figures, where showings therein are for purposes of illustrating embodiments of the present disclosure and not for purposes of limiting the same. The components in the drawings are not necessarily to scale, emphasis instead being placed upon clearly illustrating the principles of the present disclosure.

FIG. 1. illustrates an example concrete monitoring and reporting system in accordance with one or more embodiments of the present disclosure.

FIG. 2. illustrates example logger and reader devices in accordance with one more embodiments of the present disclosure.

FIG. 3 illustrates components of an example curing box in accordance with one or more embodiments of the present disclosure.

FIG. 4 illustrates a wireless box in accordance with one or more embodiments of the present disclosure.

FIG. 5 illustrates an example cloud application processing system in accordance with one or more embodiments of the present disclosure.

FIG. 6 illustrates an example process for operating the system of FIG. 1, in accordance with one or more embodiments of the present disclosure.

### DETAILED DESCRIPTION

Various embodiments of the present disclosure improve monitoring and analysis of concrete and other construction materials. Users may access and analyze real-time data from remote locations, including strength and temperature data from concrete placed in any construction location. The systems and methods disclosure herein improve the speed of decision making processes providing users with greater flexibility to avoid quality issues and take corrective action. The system includes real-time analytics and alerts to keep users informed of events and selected data milestones.

In various embodiments, the systems and methods disclosed herein include interfaces and modules facilitating user adjustment of comparison points for multiple loggers by lining up start times or other events with an interactive interface feature (e.g., sliding scale allowing the user to drag an indicator and/or a datapoint, providing a data visualization for each of a plurality of loggers and sliding the visual representation or an indicator to align the data, and/or other interactive features). The systems may be configured with improved data visualization tools, such as data from loggers which are configured to periodically store temperature and a current maturity value (e.g., every minute for 180 days).

The system may be further configured to allows users to measure and document temperature profiles and regulate thermal protection. In this regard, the system is configured to monitor optimal temperature differentials using real-time temperature information, ensure adherence to temperature specifications by logging max/min temperatures for a construction application, and/or expand construction calendar and provide effective management of fuel costs in cold weather. The system may be further configured to speed up concrete operations and extend the construction calendar in cooler weather, which may include being configured to document early strength and temperature information, improve quality control with early validation of batching consistency, and/or decrease labor and material costs.

In various embodiments, the system is configured to sense and track concrete related data at a project site and load the data to a cloud application. The data upload may be facilitated in real-time, periodically (e.g., every minute for 180 days), manually via user controls, and/or through other procedures. The cloud application facilitates near real-time data sharing of critical concrete information, including temperature, strength and differential comparisons (e.g., comparing the temperatures of a set of loggers all of which are valuable to the user). The user can access this information from a remote device, such as a smartphone, tablet and/or personal computer. The system can also be configured to notify the user based on various settings in the cloud software via text, email or other notification protocol.

The systems disclosed herein provide secure, uninterruptable, and unalterable data. The system may include tools such as multiple mix design access, alert settings, and an infrared camera for temperature measurement and heat profiling. The cloud application includes interfaces for reporting and reviewing critical data from a remote device, such as a computer or mobile device via secure cloud-based software. The system may use embedded loggers that calculate, measure and store maturity and temperature along with other planned information from sensors that are embedded in the concrete.

Additional benefits of the disclosed systems and methods include access the latest strength and temperature data from any location, avoiding quality issues by giving the user notice and data to determine and take corrective action early. The notice may be provided to keep users informed by sending reports and alerts via email, and interactive visual interfaces allow the user to adjust comparison points of multiple loggers that concrete hits at variable times by lining up (e.g., the start times with a sliding-scale feature) two or more data streams. The users may see more data than is available through conventional systems, including data received from loggers that store temperature and the current maturity value periodically (e.g., every minute). The users may access the latest strength and temperature data from any location, and the system may further associate thermal images with logger temperature data and physical jobsite location. The thermal image data may be analyzed (e.g., through thermal image analysis) to provide additional data points on the thermal properties of the concrete. The interface may be configured to associate thermal images with logger temperature data and jobsite location (e.g., by assigning a timestamp or other temporal characteristic to the thermal image, location identifier and/or an associated logger) and display the thermal image and/or associated logger data to the user. In one embodiment, the thermal image is displayed in a window and the user may scroll through thermal images (e.g., forward and backward in time) to see changes over time, along with temperature and/or other data related to the site.

In other embodiments, sensors for detecting other critical values such as humidity, corrosion, strain and other critical values and incorporate that information into the system/cloud software are provided. The system may be able to communicate directly from the sensor to a tablet, smart phone or other mobile device, and then to the cloud software—rather than requiring the connection to a wireless box. Other embodiments may incorporate other systems to show the location of sensors such as CAD drawings of jobsites and other visual representations of logger locations. Other embodiments may use logger data to extrapolate temperature information to create a visual representation of a structure's temperature profile or history.

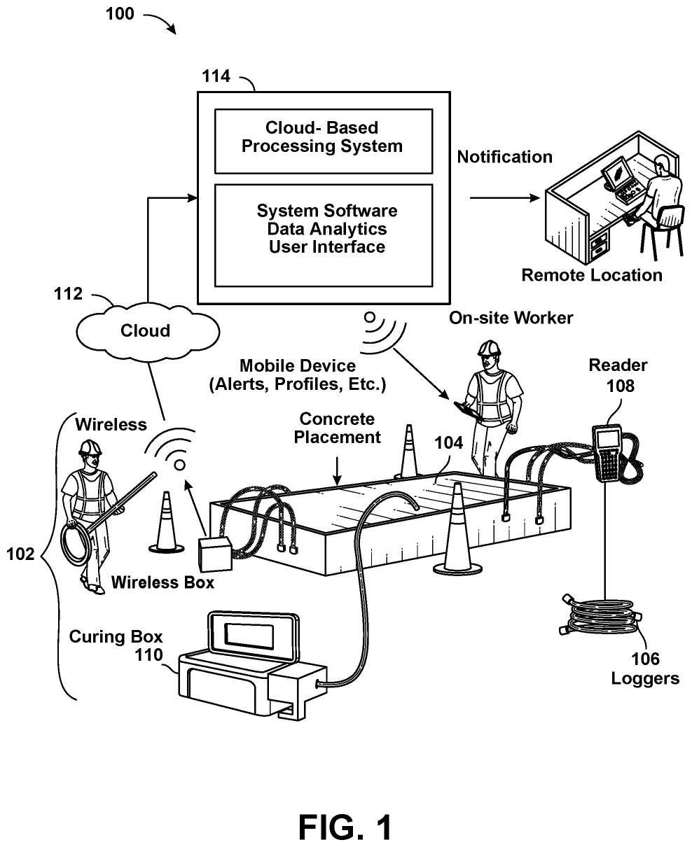

Referring to FIG. 1, a data monitoring, analysis and reporting system will now be described in accordance with one or more embodiments of the present disclosure. A system 100 includes a local site 102 (e.g., a construction site) including concrete 104. The local site 102 includes one or more loggers 106, which are configured to receive sensor and/or non-sensor data associated with the concrete 104, store the received data, and transfer the data to a separate device. Sensor data may be received via communication with a sensor (e.g., contained within the logger and/or implemented as a separate device). Non-sensor data may be received, for example, via communication with a user device (e.g., user configuration of the logger).

The logger 106 is configured to be be securely anchored in a given location, which may involve embedment or attachment of the logger into or onto an immovable (or difficult-to-move) object, such as a concrete mass (e.g., concrete 104). The concrete mass could be a concrete slab of a building, a concrete column on a bridge, a concrete pavement, a concrete vault wall, or any number of other uses of concrete. The logger can be embedded into the concrete by placing it into the concrete forms prior to placement of the concrete itself, or the logger can be physically placed into the concrete mass while the concrete remains in a non-hardened state. In other embodiments, the logger can be attached such as via a welded-encasement to or within a metal wall, a steel I-beam or other difficult-to-move object.

The logger 106 may be used, for example, to measure and record the internal temperature history of the concrete 104 and/or to collect other sensor data. Given the curing temperature history, the system 100 can calculate and record the mechanical strength of the concrete mass at a given time. A reader 108 located outside the concrete mass facilitates user interaction with the logger data, including visualization of the curing temperature history. A communications device, such as wireless box, facilitated communications between the loggers 106 and a cloud-based processing system 114 through a network 112.

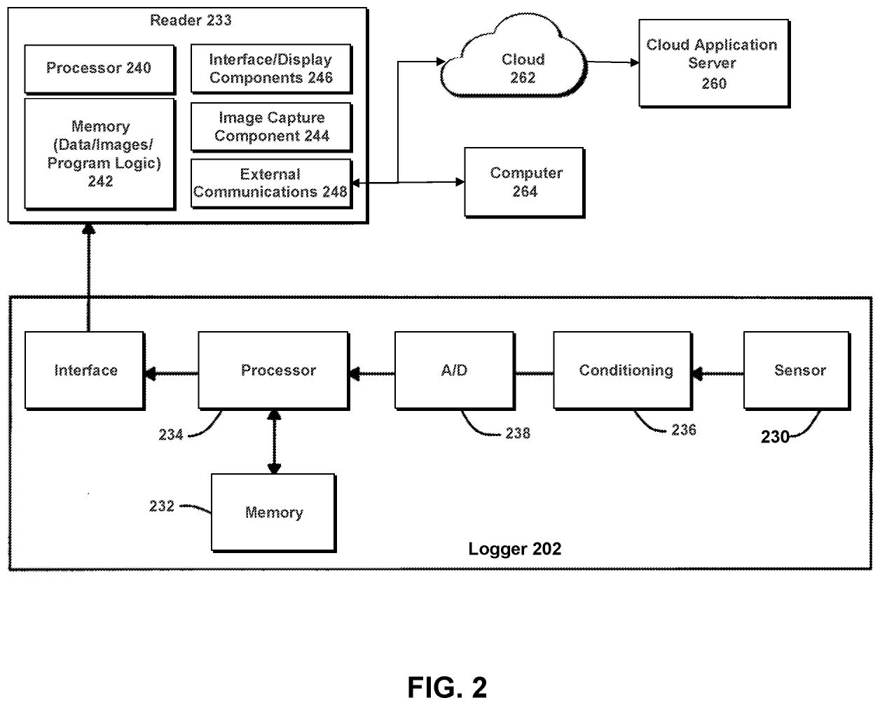

Referring to FIG. 2, embodiments of a logger 202 and reader 233 will now be described. The logger 202 can be a data logger that is adapted to record at least one data input. The logger 202 can be partially or completely housed inside a control module housing. The logger 202 can be adapted to log temperature and/or other data from a sensor 230 (e.g., a temperature sensor), which can be located within the control module housing and/or located separate from the control module housing. The logger 202 can be a sensor/recorder device which monitors and logs certain variables relating to the concrete 104, such as temperature, pH, etc. The data can be extracted from the logger 202 and used to assess the compliance of a particular process batch with applicable ASTM or other standards, or contractor requirements. In order to ensure quality control, the data recorded in the logger 202 can be encrypted and/or unalterable once recorded by the logger 202. The data can be extracted and/or transmitted to an external device, such as the reader 233 or a wireless box, and sent to a remote location (e.g., a cloud server) and decrypted/verified by an independent quality control entity and/or the project managers. Exemplary embodiments of a reader 233 are described in U.S. Pat. No. 6,865,515, which is incorporated herein by reference.

The logger 202 can save data in a memory and/or can transmit and/or receive data to the reader 233 wirelessly and/or over a network such as a LAN or the Internet via a wireless or wired transceiver. Additionally, the logger 202 may also receive and log data from thermometers and/or humidity sensors embedded in the concrete. The logger 202 may be powered by one or more of a battery, a solar cell, a portable generator, or by the electrical grid in order to provide for uninterrupted data logging throughout the curing process. Power supply redundancy can be built in for example by securing one primary and one or more secondary power sources for the logger 202. Examples of a suitable logger 202 are described in U.S. Pat. No. 6,865,515.

In the illustrated embodiment, the logger 202 includes at least one sensor 230 (e.g., a temperature sensor), a memory device 232, one or more processors 234, and a power source. The memory device 232 may, be one of several memory devices, such as a RAM device within a computer, flash memory, or a EEPROM. Within the logger 202, the sensor 230 is communicably coupled to the processor 234 (e.g., through a signal conditioning circuit 236 and/or an Analog to Digital (A/D) converter 238). The sensor 230 may be implemented as a temperature sensor, such as a thermistor for which the electrical resistance changes in an electrical circuit based on the changes of temperature sensed. The temperature sensor sends temperature dependent data signals to the processor 234 for processing. The processor 234 may be any processor, such as one or more of a microprocessor, a CPU, one or more FGPA, a microcontroller, and/or combinations thereof, for example. The logger 202 may be housed inside a control module housing. It is to be understood that more than one logger 202 may be used with the systems and methods disclosed herein.

In one embodiment, the logger 202 includes a maturity logger configured to analyze the time and temperature profile of in-place concrete to calculate its strength in real-time. The maturity loggers may be based on ASTM C 1074 “Standard Practice for Estimating Concrete Strength by the Maturity Method.” In one embodiment, the logger stores the current maturity and temperature value every minute for 180 days. A temperature logger may be configured to measure and store temperature every minute for 180 days.

The reader 233 is operable to interface with the logger 202, a cloud application server 260, and other devices. In the illustrated embodiment, the reader 233 includes a processor 240, a memory 242, an image capture component 244, an interface/display component 246 and/or external communications components 248. The reader 233 may be implemented, for example, as a hand held mobile device for use by personnel at a construction site to configure and/or control the logger 202, receive data from the logger 202 and/or other devices at the project site, and access a cloud application and/or cloud storage through the cloud application server 260. The image capture component 244 may include an infrared imaging device, a visible light imaging device (e.g., a video camera), or a multi-band imaging device for capturing and processing images, such as video images of a scene.

In various embodiments, processor 240 may comprise any type of a processor or a logic device (e.g., a programmable logic device (PLD) configured to perform processing functions). Processor 240 may be adapted to interface and communicate with various components of the reader 233 to perform method and processing steps and/or operations, as described herein. Memory 242 includes one or more memory devices adapted to store data and information, including for example sensor data from one or more loggers, thermal image data, and/or other data. Memory 242 may comprise one or more various types of memory devices including volatile and non-volatile memory devices. In one aspect, the memory 242 comprises a random-access memory (RAM), a read-only memory (ROM), component electronically programmable read-only memory (EPROM), erasable electronically programmable read-only memory (EEPROM), other flash memory, Secure Digital (SD) Card, as well as other suitable forms of memory. The functions of the reader 233 may be implemented through dedicated hardware and circuitry and software programs that may be installed into the memory 242. The reader 233 generally includes several software programs or modules, each comprising a plurality of executable instructions which, when stored in the memory 242, cause the processor 240 to perform the processes shown and described hereinafter.

In various embodiments, the processor 240 comprises an embedded microprocessor for data processing as well as controlling the operation of the reader 233. Generally, the embedded microprocessor comprises a plurality of digital and analog ports for interfacing with the different components of the reader 233. In one aspect, the microprocessor controls the image capture component 244, display component 246 and external communications components. In one aspect, the embedded microprocessor comprises a system-on-chip as well as a digital signal processing (DSP) architecture, peripherals for interfacing with the different components in the reader 233, peripherals for networking, booting and encryption, and may run an operating system.

The reader 233 may be programed via external communications components 248 to perform various aspects of the present disclosure, and any resulting software programs are generally stored in the memory 242. In one aspect, the microprocessor commands the image and wireless sensors to acquire data from an associated area, processes the data from the different sensors, and outputs analytical results according to the various embodiments of the present disclosure. The reader 233 may also comprise ports of power delivery, programing, data transfer, networking and any other component as required by the processes described by the various embodiments of the present disclosure. In the illustrated embodiment, the external communications components 248 are configured to facilitate communications with a computer 264 (e.g., a laptop computer) and a cloud application server 260 through a cloud network 262.

Image capture component 244 comprises, in one embodiment, any type of image sensor operable to detect and track conditions of relevance to the construction project. For example, the image capture component 244 may include an image sensor having one or more image detector elements such as infrared (e.g., thermal) photodetector elements and/or visible light photodetector elements for capturing infrared image data (e.g., still image data and/or video data) representative of a scene such as an image of the concrete. In one embodiment, image capture component 244 may be configured to generate digital image data representing incoming thermal radiation from the concrete. Image capture component 244 may include one or more signal processing components such as analog-to-digital converters included as part of an image sensor or separate from the image sensor as part of reader 233. In one aspect, image data (e.g., video data) may comprise non-uniform data (e.g., real image data) of the concrete. Processor 240 may be adapted to process the image data (e.g., to provide processed image data), store the image data in memory 242, and/or retrieve stored image data from memory 242. For example, processor 240 may be adapted to process image data stored in memory 242 to provide processed image data and information (e.g., captured and/or processed image data).

The interface/display components 246 comprise, in one embodiment, a user input and/or interface device. For example, the user input and/or interface device may represent a rotatable knob, push buttons, slide bar, keyboard, etc., that is adapted to generate a user input control signal. Processor 240 may be adapted to sense control input signals from a user via interface/display components 246 and respond to any sensed control input signals received therefrom. Processor 240 may be adapted to interpret such a control input signal as a parameter value, as generally understood by one skilled in the art. In one embodiment, interface/display components 246 may comprise a separate control unit (e.g., a wired or wireless unit) having push buttons adapted to interface with a user and receive user input control values. In one implementation, the push buttons of the control unit may be used to control various functions of the reader 233.

The interface/display components may include an optional display component which comprises, in one embodiment, an image display device (e.g., a liquid crystal display (LCD) or various other types of generally known video displays or monitors). Processor 240 may be adapted to display image data and information on the display component. Processor 240 may be adapted to retrieve image data and information from memory 242 and display retrieved image data and information on display component. Display component may comprise display electronics, which may be utilized by processor 240 to display image data and information (e.g., infrared images). In some embodiments, display components may be provided through other user devices (e.g., a mobile device or desktop computer) that access processed data via a network or server system.

In various embodiments, components of reader 233 may be combined and/or implemented, as desired or depending on the application or requirements, with other components of the system. Furthermore, various components of reader 233 may be remote from each other (e.g., image capture component 244 may comprise a remote sensor with processing component and other components. The hardware and specific circuitry of embodiments of the reader 233 can vary according to aspects of the present disclosure. Accordingly, although examples of the hardware associated with the reader 233 have been previously described, it is not intended that the present disclosure be limited to the specific embodiments or aspects presented, and other variations of the reader 233 and logger 202 will be apparent to one of ordinary skill in the art.

External communication components 248 can include a variety of suitable input/output connection interfaces, such as wired connections, standard serial ports, parallel ports, S-video ports, large area network (LAN) ports, small computer system interface (SCSI) ports, or other suitable wired connections. Additionally, the external communication components 248 can include, for example, wireless connections, such as 802.11p, infrared ports, optical ports, Bluetooth wireless ports, wireless LAN ports, ultra-wide band (UWB) wireless ports, among others as will occur to one of ordinary skill in the art.

Referring back to FIG. 1, a curing box 110 is configured to receive one or more concrete test containers for further testing and monitoring. The curing box 110 is configured to receive concrete test containers and operate to a set temperature inside the curing box 110. In various embodiments, the curing box 110 may be filled with liquid and heated/cooled to a desired pre-set temperature prior to inserting the concrete test containers. After the concrete has cured, the concrete test containers are removed from the curing box 110 and sent to an off-site location for quality control and/or concrete strength testing. The data recorded by a logger can also be sent to the control lab with the concrete test containers to establish proper on-site handling procedures. In various embodiments, the curing box 110 is designed to meet applicable industry standards for a jurisdiction of use and may be implemented in accordance with the curing box disclosed in U.S. Pat. No. 9,404,672, which is incorporated herein by reference.

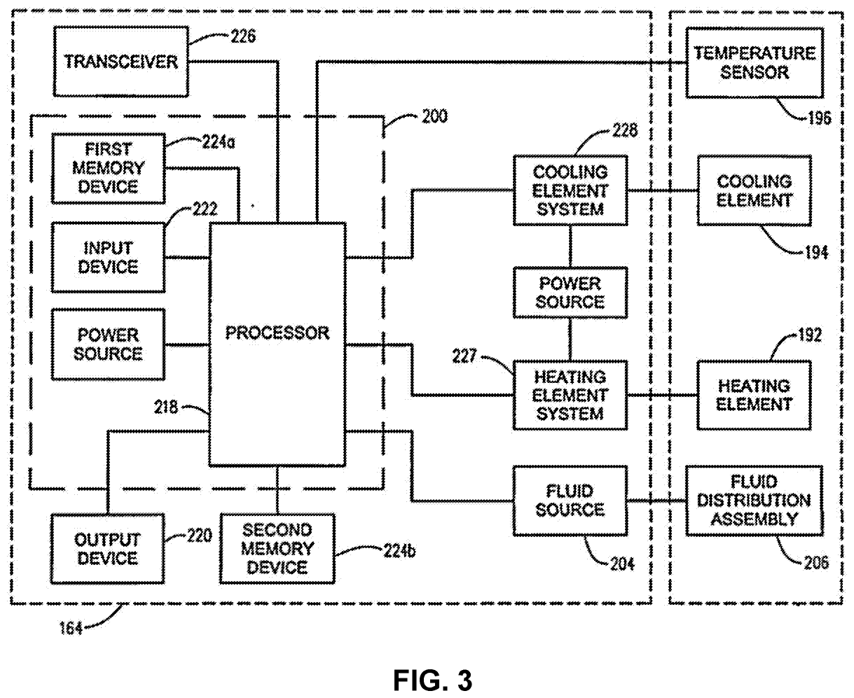

Referring to FIG. 3, an example embodiment of a control module 200 for controlling the curing box 110 is illustrated. The control module 200 is adapted to control the heating element 192, the cooling element 194, and the circulator assembly, which may comprise a fluid source 204 and a fluid distribution assembly 206. The control module 200 is further adapted to receive and/or record data from the temperature sensor 196 and/or from a logger. The control module 200 can have one or more processors 218, one or more output devices 220, one or more input devices 222, and one or more memory devices 224. The one or more processors 218 may be any processor known in the art such as one or more of: a Central Processing Unit (CPU), a microprocessor, a Field Programmable Gate Array (FGPA), or the like, or combinations thereof, for example. The one or more processors may function to control the operation of a temperature control assembly and/or a circulator assembly. The one or more memory devices 224 can be a hard drive, a solid-state drive, Random Access Memory (RAM), a flash memory, a floppy drive, or an Electronically Erasable Programmable Read-only Memory (EEPROM), or combinations thereof, for example. The one or more memory devices 224 can include a first memory device 224a and a second memory device 224b. The second memory device 224b can be a removable memory device such that the second memory device 224b can be sent off-site to a quality control lab, for example, such that the quality control lab can access and verify data contained in the second memory device 224b. The one or more memory devices 224 can store computer executable code and/or other data. The computer executable code stored in the one or more memory devices 224 can be accessed by the one or more processor 218 to obtain the data stored therein in situ, or over a network such as a Local Area Network (LAN), wireless network, or the Internet. Data contained in the one or more memory devices 224 may also be remotely accessible over a network such as LAN, a cellular phone network, a Wi-Fi network, or the Internet, for example, by using a transceiver 226. The control module 200 can monitor the temperature inside the curing box 110 via the temperature sensor 196 in real time and adjust the heating element 192 and/or the cooling element 194 accordingly via a heating element system 227 and/or a cooling element system 228. The heating element system 227 can be provided with relays, circuitry and/or controllers to adjust the heat being generated and/or absorbed by the heating element 192. The cooling element system 228 can function based upon the principles of absorption, heat pump cycles, or refrigeration cycles, and may include components such as one or more compressor, heat pump, condenser, expansion valve, evaporator, controller and/or other devices and/or circuitry working together to adjust the heat absorption of the cooling element 194.

The output device 220 can be a Light Emitting Diode display (LED), a Liquid Crystal Display (LCD), a touchscreen display, an analog temperature gauge, or a digital temperature gauge, for example. The control module 200 can produce audible and/or visible alert signals when the temperature inside the curing box 110 varies from a pre-set value and/or tolerance, such as 73° F.±3° F., for example. The alert signal can be displayed such that it is visible to a worker observing the curing box 110, or can be transmitted to a reader, cloud application server and/or another remote location. A user may manually adjust the temperature inside the curing box 110 via the input device 222 or pre-set a temperature to be automatically maintained by the control module 200 inside the curing box 110. The processor 218 may also execute instructions to enable a user to adjust and/or monitor the temperature inside the curing box 110 remotely over a network such as a wireless network, a LAN, a telephone network, the Internet, and/or a cellular network. For example, the processor 218 can be programmed to host a web-site having a Uniform Resource Locator and/or an IP address accessible via the Internet to permit the user to adjust and/or monitor the temperature. Further, the processor 218 may receive a temperature log from an in-situ specimen and/or the input device 222 and/or a remote network and control the temperature inside the curing box 110 to substantially correspond to the temperature log.

In accordance with the present disclosure, a match temperature sensor, such as a logger 202, can be mounted in or on an in-situ curing concrete mass, such as a road, while one or more concrete test specimens are cured within the box 110 in a wet environment. The control module 200 is programmed and operated to cause the temperature within the fluid of the box 110 (as read by the temperature sensor 196) to substantially correspond to the temperature of the in-situ concrete mass read by the match temperature sensor. Thus, in the present disclosure, the temperature of the concrete test specimens is regulated indirectly in a wet environment via temperature readings and temperature control of the fluid in a new and inventive manner.

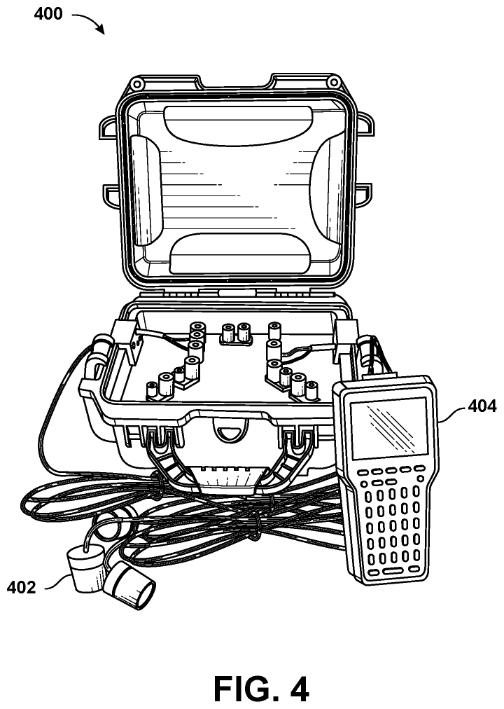

Referring to FIG. 4, a wireless box 400 connects one or more loggers 402 to a network interface, allowing access of data from a computer, wireless or web-enabled device. Data may be downloaded from the loggers directly to the cloud software system, straight from the concrete placement. With each concrete placement, there are critical strength and temperature targets that may be tracked and met. Construction sites further have numerous people, heavy equipment and machinery which can damage monitoring equipment. The wireless box 400 is configured to withstand the elements of construction site and reliably provide data (temperature and strength) at any time of day at any location. The wireless box 400 may be configured to provide up-to-the minute concrete monitoring from remote locations, any time of day. In the illustrated embodiment, the wireless box 400 is configured to communicate with a cloud-based processing system 114, allowing the user to review and report latest concrete data, including differential alerts. Use of the system allows the user to make corrective actions early. The reader 404 includes an on-board thermal imager for temperature profiling and problem detection. The data is stored within the logger 402, so it cannot be altered. In one embodiment, the wireless box 400 is configured to provide immediate data transfer to the cloud-based processing system 114.

The cloud-based processing system 114 may include data storage and processing capabilities as described herein. In some embodiments, the cloud-based processing system 114 is configured to provide user-defined alerts and alarm conditions to users when events occur. The software allows the user to view real-time concrete strength and temperature data from a computer or mobile device. The software is configured to send data directly from the loggers to the cloud software via wireless communications interface (e.g., cellular LTE 4G wireless). The software may also be configured to set up differential alerts to users (e.g., view email, text message or other alert protocol), create and send reports, see calibration curves, monitor the temperature profile of multiple loggers, and compare surface and internal temperature information by analyzing thermal images.

In various embodiments, the cloud-based software allows the system to review and report critical strength or temperature data on a mobile device or computer, allow the user to make informed decisions based on real-time strength and temperature data, and set up alerts to monitor concrete temperature remotely with periodic temperature values delivered per project specification, and meet remote notification demands with user-defined alerts.

In various embodiments, the cloud software allows a user to view up-to-the-minute concrete data (e.g., strength data, temperature data, calibration curves, logger data, thermal images, etc.). The system allows the user to set up customer alerts such as target strength values (e.g., PSI) and messages. In one embodiment, the user may set up an alert, including an alert threshold for triggering the alert. By providing real time monitoring, the cloud-based system can help prevent thermal cracking by monitoring differential temperatures and receiving timely alerts when conditions are triggered. The user can receive reliable, valid data from start to finish, and ensure strict specifications are met, detect delamination, temperature gradients, temperature extremes, and other monitoring functions.

A handheld reader (e.g., reader 108) may be provided to assist on-site personnel in managing, configuring and accessing local loggers and systems. The reader can be used, for example, to start the loggers and provide the user with a display to view critical numeric and graphed data in the field. In some embodiments, the reader includes a built-in thermal imager, storage for data from thousands of loggers and other features. The reader provides connectivity to loggers in the field so critical decisions can be made at the touch of a button. A user can activate loggers to start tracking key data points during concrete curing. The built-in thermal imager allows for temperature profiling and onsite problem detection. The visual display of the thermal data allows the user to identify surface temperature issues and monitor insulation performance or cylinder curing conditions. The reader may be used to input important notes and jobsite or location information specific to that logger. The user may also be able to view stored temperature and strength data and record important notes to keep the project on track, cut the time spent using equipment rentals and reduce cement and labor costs.

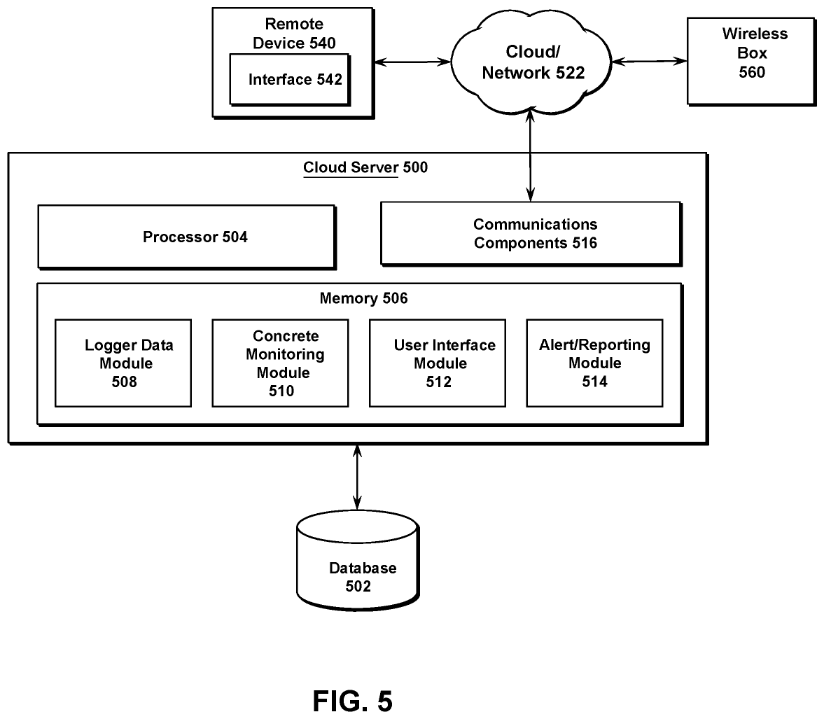

An embodiment of a cloud server 500 will be further described with reference to FIG. 5. The cloud server 500 includes a database 502, a processor 504, a memory 506 and communications components 516. The database 502 stores sensor data, logger data, analysis results and other data used by the concrete monitoring and reporting system. The processor 504 may include one or more processors or controllers for controlling the operation of the cloud server 500, which may include executing instructions stored in the memory 506. The memory 506 may include random access memory, read only access memory and/or other memories for storing data and program instructions. In some embodiments, the memory 506 stores one or more applications including a logger data module 508 for retrieving, storing and organizing logger data, a concrete monitoring module 510 configured to track characteristics of monitored concrete, a user interface module 512 providing a user interface allowing visualization of the concrete data, and an alert/reporting module 514 configured to detect and generate alert conditions and reports. The cloud server 500 may enable a number of different applications which include but not are limited to the applications described herein. The communications components 516 facilitate communications with one or more network devices through a network 522 such as the cloud and/or internee. The devices may include a remote device 540 configured to display a user interface 542 as disclosed herein (e.g., a reader, a personal computer, a mobile device), a wireless box 560 providing communications between the cloud server 500 and one or more loggers, and/or other devices.

The data available in the cloud server 500 may be used to identify and detect certain events. The events may be system generated and/or user generated based on a comparison of data conditions with a threshold value. The cloud server 500 may also provide the ability to automatically review logs as uploaded by the collection subsystem to identify misconfigured sensors or other issues which may be indicative of improper setup or sensor failure. By monitoring logs in aggregate, advance notice may be given to end users regarding likely system maintenance or potential system failure. The cloud server 500 may allow users to analyze historical information and/or monitor concrete characteristics in real time.

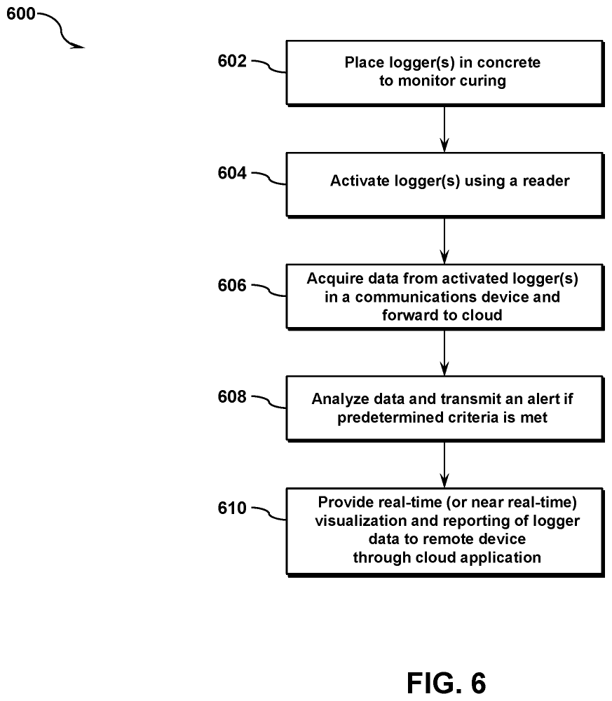

An example operation of a system in accordance with one or more embodiments of the present disclosure will now be described with reference to FIG. 6. A process 600 may be implemented by software instructions executing on one or more devices of the concrete monitoring and reporting system. In step 602, one or more loggers are placed in concrete to monitor curing. The loggers may be configured, activated and read by a reader in step 604. After activation, the loggers are configured to periodically sense certain concrete conditions (e.g., temperature) and store the data in a memory. In step 606, a communications device (e.g., wireless box of FIG. 1) acquires data from the loggers at a construction site and forwards the data to the cloud service. In various embodiments, the loggers are configured to periodically record data (e.g., every minute) and the communications device is configured to upload data to the cloud in real-time or near real-time manner. In step 608, the cloud application analyzes the received data, and determines whether a tracked event has occurred. For example, a user may set a threshold to trigger an alert when the threshold is exceeded. An alert may be sent to one or more recipients indicating that the event has occurred. The alert may be sent using any suitable communication protocol including email, texting, a voice call playing a computer-generated message, a notification through a mobile application or other messaging approach. In step 610, the cloud service provides one or more users with real-time access to available data through a cloud application. In various embodiments, the users may access the data through a web interface, a mobile application, an application running on a computer, or through other approaches. The cloud application is configured to provide access to available data, analytics, visualizations, system configurations and customizations, and other services.

Where applicable, various embodiments provided by the present disclosure can be implemented using hardware, software, or combinations of hardware and software. Also, where applicable, the various hardware components and/or software components set forth herein can be combined into composite components comprising software, hardware, and/or both without departing from the spirit of the present disclosure. Where applicable, the various hardware components and/or software components set forth herein can be separated into sub-components comprising software, hardware, or both without departing from the spirit of the present disclosure.

Software in accordance with the present disclosure, such as non-transitory instructions, program code, and/or data, can be stored on one or more non-transitory machine-readable mediums. It is also contemplated that software identified herein can be implemented using one or more general purpose or specific purpose computers and/or computer systems, networked and/or otherwise. Where applicable, the ordering of various steps described herein can be changed, combined into composite steps, and/or separated into sub-steps to provide features described herein. Embodiments described above illustrate but do not limit the invention. It should also be understood that numerous modifications and variations are possible in accordance with the principles of the invention. Accordingly, the scope of the invention is defined only by the following claims.

1. A system comprising:
communications components configured to receive concrete data from a plurality of sensors associated with a concrete mass and adapted to measure a property of the concrete mass, the concrete data comprising a plurality of data streams containing measurement data for the property from respective different sensors;
a storage device configured to store the received concrete data; and
a logic device configured to:
detect an event in the concrete data;
transmit a notification of the detected event to at least one remote device; and
render an interface for the remote device, the interface comprising one or more interface features configured to facilitate visualization of the stored concrete data by allowing a user to adjust comparison points of different data streams and align the different data streams based on the comparison points.

2. The system of claim 1, wherein the communications components are configured to receive the data streams from a reader device and distribute the data streams to the at least one remote device.

3. The system of claim 1, wherein each sensor is part of a logger which also comprises:
a logic device configured to receive the sensor's data and calculate data for the respective data stream; and
a data transfer device configured to transmit the respective data stream to an external device;
wherein adjusting the comparison points comprises aligning start times of concrete monitoring processes.

4. The system of claim 3, further comprising a reader configured to receive the respective data stream from the data transfer device of the corresponding logger;
wherein the one or more interface features comprise a sliding scale allowing the user to drag an indicator and/or a datapoint.

5. The system of claim 4, wherein the reader comprises an infrared imaging component configured to capture an infrared image of the concrete mass;
wherein the concrete data includes the captured infrared image; and
the concrete mass is at least part of a building, a bridge, a pavement, or a wall, and at least two of the sensors are embedded in the concrete mass.

6. The system of claim 3, wherein the logger is further configured to store non-sensor data comprising location information, a project identifier, time-related data, mix design information, temperature, activation energy and/or a threshold value; and
the concrete mass is at least part of a permanent structure.

7. The system of claim 1, wherein the measurement data comprises temperature, moisture, relative humidity, electrical conductivity, thermal conductivity, alkalinity, oxidation-reduction potential and/or sonic velocity; and
wherein the interface provides a data stream visualization for each of the plurality of sensors and allows sliding the visual representation or an indicator to align the data streams.

8. The system of claim 1 wherein the storage device is a cloud storage device and wherein the logic device comprises a cloud application server.

9. The system of claim 1, wherein each sensor is part of a logger configured to capture and store sensed data every minute and wherein the communications components are configured to receive the concrete data after the concrete data is available from the logger, thereby providing a remote device with up-to-the minute access to the concrete data.

10. The system of claim 1, wherein each sensor is part of a logger, and the interface comprises an interactive control facilitating user adjustment of comparison points for multiple loggers.

11. A method comprising:
receiving, through communications components, concrete data from a plurality of sensors associated with a concrete mass and adapted to measure a property of the concrete mass, the concrete data comprising a plurality of data streams containing measurement data for the property from respective different sensors;
storing the received concrete data in a storage device;
detecting an event in the concrete data;
transmitting a notification of the detected event to at least one remote device;
rendering an interface for the remote device, the interface comprising one or more interface features configured to facilitate visualization of the stored concrete data; and
in response to a user operating at least one of the one or more interface features, adjusting comparison points of different data streams and aligning the different data streams based on the comparison points.

12. The method of claim 11, further comprising receiving the data streams from a reader device and distributing the concrete data streams to the at least one remote device.

13. The method of claim 11, further comprising:
receiving, by a logger comprising a sensor which is one of said sensors, the sensor's data and calculating data for the respective data stream; and
transmitting the respective data stream from the logger to an external device;
wherein adjusting the comparison points comprises aligning start times of concrete monitoring processes.

14. The method of claim 13, further comprising receiving at a reader device the respective data stream from a data transfer device of the corresponding logger;
wherein the one or more interface features comprise a sliding scale allowing the user to drag an indicator and/or a datapoint.

15. The method of claim 13, further comprising capturing an infrared image of the concrete mass; wherein the concrete data includes the captured infrared image;
wherein the concrete mass is at least part of a building, a bridge, a pavement, or a wall, and at least two of the sensors are embedded in the concrete mass.

16. The method of claim 13, further comprising storing non-sensor data on the logger including location information, a project identifier, time-related data, mix design information, temperature, activation energy and/or a threshold value;
wherein the concrete mass is at least part of a permanent structure.

17. The method of claim 11, wherein the concrete data comprises temperature, moisture, relative humidity, electrical conductivity, thermal conductivity, alkalinity, oxidation-reduction potential and/or sonic velocity; and
wherein the interface provides a data stream visualization for each of the plurality of sensors and allows sliding the visual representation or an indicator to align the data streams.

18. The method of claim 11 wherein the storage device is a cloud storage device and wherein the method further comprises a cloud application interface.

19. The method of claim 11, wherein each sensor is part of a logger, the method further comprising:
capturing and storing sensed data every minute; and
receiving concrete data at a cloud service after the concrete data is available from the logger, thereby providing a remote device with up-to-the minute access to the concrete data.

20. The method of claim 11, wherein each sensor is part of a logger, and the interface comprises an interactive control facilitating user adjustment of comparison points for multiple loggers.
